(19) United States
(12) Patent Application Publication
(10) Pub. No.: US 2009/0024094 A1
(43) Pub. Date: Jan. 22, 2009

(54) Device for injecting an injectable product

(76) Inventors: Peter Eichhorst, Hohen-Neuendorf (DE); Wolfgang Heidl, Muennerstadt (DE)

Correspondence Address: Millen, White, Zelano & Branigan, Arlington Courthouse Plaza 1, 2200 Clarendon Blvd. Suite 1400, Arlington, VA, 22201, US

(21) Appl. No.: 10/569,626

(22) PCT Filed: Aug. 23, 2004
(86) PCT No.: PCT/EP2004/009618

§ 371 Date: May 21, 2007

(30) Foreign Application Priority Data
Aug. 29, 2003 (DE) 103 40 613.1

(51) Int. Cl. A61M 5/315

(52) U.S. Cl. 604/218

(57) ABSTRACT

The invention relates to a device (10) for injecting an injectable product, comprising a reservoir (12), which holds the product, a discharge assembly (14) for discharging the product out of the reservoir (12), and an actuating device (16) for subjecting the product inside the reservoir (12) to a pressure. The invention provides that the device (10) can be used as a single-use syringe or as an ampoule for a needleless injector.

The invention relates to a device for injection of an injectable product, with a storage receptacle that holds the product, a withdrawal arrangement for transferring the product out of the storage receptacle, and an actuation device for applying pressure to the product in the storage receptacle.

Devices of the generic type are known. The latter are used for injection of an injectable product, the injectable product being defined in particular as liquids, for example solutions, suspensions, or dispersions that contain an active substance. Active substances can be pharmacologically active substances for treatment of the human or animal body or substances for diagnosis or for cosmetic use. Furthermore, the injectable products can also be used to deliver active substances to plants.

Devices of the generic type for subcutaneous, intravenous, intramuscular or intracutaneous administration of injectable products are known. Here, devices are known in which the administration of the injectable product takes place by injection using a needle. Here, so-called disposable syringes are known that encompass a storage receptacle that can be filled beforehand with the product to be injected, which is connected to a needle covered by a protective cap, and to which, in contrast, an actuating means is assigned by means of which a plunger within the storage receptacle can be moved. To use this disposable syringe, the latter is removed from a sterile blister pack, the protective cap is removed from the needle, and after placing the disposable syringe in the area of the body to be addressed, the product to be injected is transferred into the tissue by pressing on the actuating means.

In contrast, so-called needle-free injectors are known, by means of which a product to be injected is applied under pressure to an area of tissue so that the product enters the tissue as a result of pressure. Such needle-free injectors have a receiving area for a prefilled ampoule that contains the product that is to be injected, an actuating means for applying pressure to the product, so that the product emerges through a nozzle arrangement and penetrates the desired tissue areas. The pressure can be applied here by a trigger element that can be pretensioned against the force of a spring element or by an external pressure medium.

Whether a disposable syringe or a needle-free injector is used depends on, among others, the effect that is intended with the product to be injected. Depending on whether the product is to be administered, for example, subcutaneously, intravenously or intramuscularly, either a disposable syringe or a needle-free injector is available as an injection device.

The object of the invention is to provide a device for injection of an injectable product that is characterized by a simple structure and that enables flexible use in the administration of injectable products.

According to the invention, this object is achieved by a device with the features named in claim 1. Because the device for injection of an injectable product can be used as a disposable syringe or as an ampoule for a needle-free injector, it is advantageously possible to decide only briefly before administering the product to be injected whether the latter is administered, for example, intramuscularly or intravenously via the disposable syringe or, for example, subcutaneously via a needle-free injector. It thus becomes possible to use the same device selectively as a disposable syringe or as an ampoule.

In a preferred embodiment of the invention, it is provided that the actuating device comprises a plunger that is guided in a sealed manner into the storage receptacle and that has a proximal area that can be separated from a distal area. In this way, it is easily possible to undertake retrofitting of the actuating device for the case in which the device is to be used for injection as an ampoule for a needle-free injector. The proximal area is preferably detachably connected to the distal area of the plunger. In this way, retrofitting can take place with a simple maneuver by loosening this connection that can be, for example, a catch connection, a positive fit or the like. Furthermore, it is preferred if the proximal area is connected to the distal area via a scored site. Here, by simply applying a force to the proximal area, the plunger can be broken off at the scored site so that simple retrofitting is likewise possible.

Furthermore, in a preferred embodiment of the invention, it is provided that the withdrawal arrangement for transferring the injectable product out of the storage receptacle encompasses a needle whose distal area can be removed in a defined manner from a proximal area of the needle. In this way, the injection device that is present as a disposable syringe can be easily retrofitted into an ampoule for a needle-free injector. For this purpose, the needle preferably has a scored site that forms a passage when used as a disposable syringe and that forms a discharge nozzle when used as an ampoule for a needle-free injector—when the distal end of the needle has been removed.

Furthermore, in another preferred embodiment of the invention, it is provided that a protective cap is assigned to the withdrawal arrangement. This protective cap is used in a way that is known in the art for protective accommodation of the needle, especially of its ground end, and, in contrast, for sealing the storage receptacle that holds the prefilled product that is to be injected. It is preferably provided that the protective cap has scoring along which the distal end of the protective cap can be removed, while a proximal end of the protective cap remains on the storage receptacle. This results in that the proximal end of the protective cap that remains on the storage receptacle assumes a protective function of the storage receptacle, especially when it is used as a prefilled ampoule in a needle-free injector. The remaining proximal end, on the one hand, forms a self-adjusting guide for the storage receptacle in a needle-free injector and, on the other hand, a ring anchor in the nozzle area of the storage receptacle. This advantageously results in that bursting of the storage receptacle at the high pressures that occur in use as a needle-free injector is prevented.

In another preferred embodiment of the invention, it is provided that the device is suited for long-term storage of products that are to be injected, especially medications. Here, it is provided in particular that the storage receptacle consists of glass, especially borosilicate glass, at least the parts of the protective cap that come into contact with the product, and the actuating device consists of butyl rubber, and the needle consists of high-grade surgical steel.

Other preferred embodiments of the invention follow from the other features named in the subclaims.

The invention is explained in more detail below in embodiments using the related drawings. Here:

FIGS. 1 to 3 show various views of a device that is used as a disposable syringe for injection of an injectable product, and

FIGS. 4 to 10 show different views of a device that is used as an ampoule for a device that is used as a needle-free injector for injection of an injectable product.

FIG. 1 shows a device for injection of an injectable product that is labeled 10 overall. The device 10 comprises a storage receptacle 12, a withdrawal arrangement 14 and an actuating device 16. In the representation shown in FIG. 1, the withdrawal arrangement 14 is covered by a protective cap 18. The storage receptacle 12 is formed by a cylindrical hollow body 20 that has an interior space 22. The hollow body 20 consists of, for example, glass, especially borosilicate glass, but can also consist of other suitable materials, for example, such as plastic, metal or the like.

The interior space 22 is bordered, on the one hand, on the proximal end by a stopper 24 that is connected to a plunger rod 26. The stopper 24 consists of, for example, butyl rubber, while the plunger rod 26 consists of, for example, plastic. The plunger rod 26 has a distal area 28 and proximal area 30 that are connected to one another via a scored site 32. The proximal area 30 is terminated by a thumb pressure plate 34.

The protective cap 18 is made essentially rotationally symmetrical and is slipped, locked or the like onto a distal end 36 of the storage receptacle 12. For this reason, a proximal end section 38 of the protective cap 18 and the distal end 36 of the storage receptacle 12 have the corresponding shape features. A distal end 40 of the protective cap 18 is connected to the proximal end 38 via a scored site 42. The protective cap 18 consists of, for example, butyl rubber.

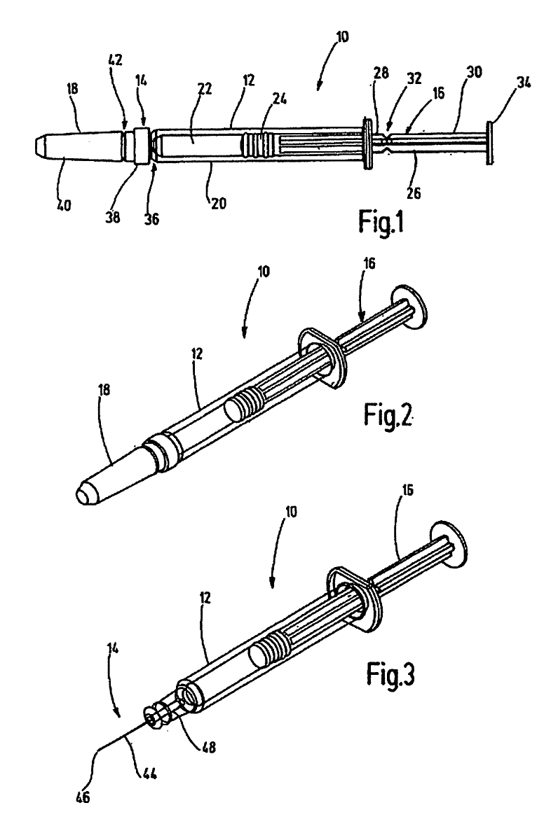

FIG. 2 shows the device 10 in a perspective view, the same parts as in FIG. 1 being provided with the same reference numbers and not being explained again.

In the perspective view shown in FIG. 3, the protective cap 18 is removed so that the withdrawal arrangement 14 is exposed. The withdrawal arrangement 14 comprises a needle 44, whose distal end has a ground section 46 and whose proximal end is inserted into a distal opening 48 of the storage receptacle 12. The needle 44 is cemented in the opening 48, for example by means of a UV-curable cement. The needle 44 consists of, for example, high-grade surgical steel.

The device 10 shown in FIGS. 1 to 3 shows the following function:

The storage receptacle 12 is filled in a way that is known in the art with the product that is to be injected, for example a solution, dispersion, suspension or the like that contains an active pharmacological ingredient, by means of a ready-to-fill filling system, and is sealed and finished with the stopper 24 and the actuating device 16. The interior space 22 thus contains a defined amount of a product that is to be injected. The needle 44 is protected via the protective cap 18, at the same time escape of the product from the interior space 22 being prevented. The device 10 is then packaged in a likewise known manner in a blister pack. By selecting the materials and packaging the device 10, long-term storage is possible.

According to the representations in FIGS. 2 and 3, the device 10 is used as a so-called disposable syringe, for example for intravenous, intramuscular administration of the product that is to be injected. To do this, the protective cap 18 is removed from the storage receptacle 12 and the product is administered as desired by means of the actuating device 16.

Based on the following FIGS. 4 to 10, the use of the device 10 shown in FIG. 1 as an ampoule for a needle-free injector is considered in detail. As already explained, the device 10 can be used according to the representation in FIG. 3 as a disposable syringe as well.

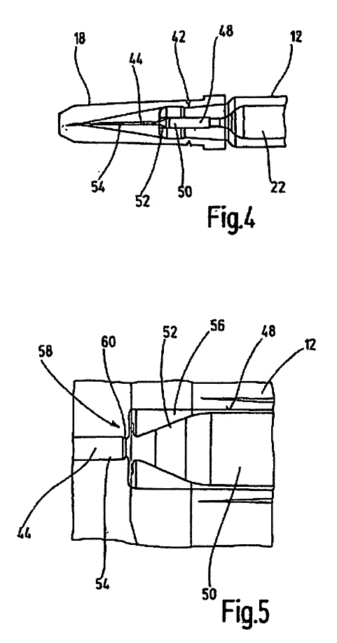

FIG. 4 shows a schematic sectional view of the device 10 in the area of its distal end, the same parts as in the preceding figures being provided with the same reference numbers. It becomes clear that the protective cap 18 surrounds the needle 44 that is cemented in the distal opening 48 of the storage receptacle 12. The needle 44 has a section 50 that lies within the opening 48 and that passes into the actual needle 44 via a conically running section 52. The section 50 here has a larger outside diameter than the section 54 of the needle 44. The section 50, for example, has an outside diameter of 1.2 mm and an inside diameter of 0.8 mm, while the section 54, for example, has an outside diameter of 0.4 mm and an inside diameter of 0.2 mm. The adaptation of the outside diameter or the inside diameter of the sections 50 and 54 takes place via the conical section 52. In a further detailed enlargement in FIG. 5, it becomes clear that the needle 44 is fixed over its sections 50 and 52 by means of a UV-curable cement 56 in the opening 48 of the storage receptacle 12. Furthermore, it becomes clear that in the transition area between the conical area 52 and the section 54 of the needle 44, there is a scored site 58. The latter is incorporated as an annular groove 60 with an open edge into the needle 44.

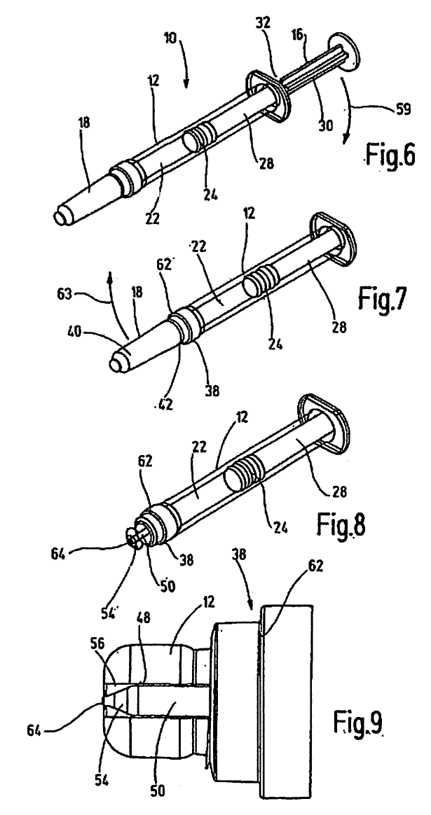

The use of the device 10 that is described in FIGS. 1 to 5 as an ampoule for a needle-free injector is described using FIGS. 6 to 10. The initial situation is the basic arrangement shown in FIG. 6 (corresponds to the representation in FIG. 2 for use as a disposable syringe). In the first step, as illustrated in FIG. 7, the proximal area 30 of the plunger rod 26 is broken off at the scored site 32 (arrow 59 in FIG. 6). This can take place by simply snapping the proximal area 30 relative to the distal area 28 of the plunger rod 26 that is located within the storage receptacle 12. The arrangement shown in FIG. 7 is thus produced. The arrangement according to FIG. 7 is inserted into the receiving area of a needle-free injector that is not shown. Here, the protective cap 18 is used as an adjustment aid by the storage receptacle 12 with the protective cap 18 being pushed into the corresponding receiving area, a collar 62 being used as a positioning stop.

Then, as illustrated in FIG. 8, the distal end 40 of the protective cap 18 is bent relative to the needle-free injector and thus to the storage receptacle 12 that is located within the needle-free injector (arrow 63 in FIG. 7). The relatively elastic protective cap 18 follows this motion. The needle 44 is broken off on its scored site 58 (FIG. 5) by this snapping motion. Then, the distal end 40 of the protective cap 18 is torn off at the scored site 42 so that the arrangement shown in FIG. 8 remains within the needle-free injector. The proximal end 38 of the protective cap 18 is used furthermore for positioning of the storage receptacle 12 within the needle-free injector. In contrast, the proximal end 38 of the protective cap 18 forms a ring anchor that encompasses the distal end of the storage receptacle 12. This again becomes clear in the enlarged representation in FIG. 9. The opening 64 that is produced at the scored site 58 of the needle 44 forms the nozzle opening for emergence of the product that is to be injected. The trigger mechanism of the needle-free injector that is not shown is accelerated to the distal end 36 of the plunger rod 26 so that the product that is to be injected and that is present in the interior space 22 is pressurized via the stopper 24. Corresponding to the suddenly occurring high pressure, the product is pressed over the proximal section 50 of the needle that has remained in the opening 48 and the conical section 54 to the nozzle opening 64 so that the product emerges with the required pressure and can be administered, for example, subcutaneously.

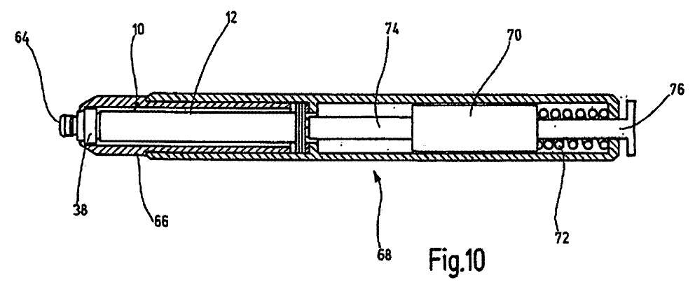

Finally, FIG. 10 again shows an overall arrangement in which the device 10 is inserted into a needle-free injector. The device 10 that has been modified accordingly in FIGS. 6 to 9 is used at this point as a prefilled ampoule for the injector.¹ In this case, the ampoules can be used in disposable or reusable injectors. ¹Can perhaps a general view of a needle-free injector with an inserted ampoule be supplied here?

Using the aforementioned explanations, it becomes clear that the device 10, as is shown in FIG. 1, can be used both as a disposable syringe (FIG. 3) and also as an ampoule for a needle-free injector (FIG. 10). With a few maneuvers, the device 10 can be retrofitted for a needle-free injector or can be used as a disposable syringe even without retrofitting. Thus, diverse, flexible possible applications of the device 10 are produced depending on the desired application. Separate production and storage of disposable syringes, on the one hand, and ampoules for needle-free injectors, on the other hand, are thus not necessary. In particular, with respect to the identical dimensions of the storage receptacle 12, important advantages arise in filling, since it is not necessary to retrofit existing ready-to-fill filling systems.

### LEGEND

10 Device
12 Storage receptacle
14 Withdrawal arrangement
16 Actuation device
18 Protective cap
20 Hollow body
22 Interior space
24 Stopper
26 Plunger rod
28 Distal area
30 Proximal area
32 Scored site
34 Thumb pressure plate
36 Distal end
38 Proximal end
40 Distal end
42 Scored site
44 Needle
46 Ground section
48 Distal opening
50 Cylindrical section
52 Conical section
54 Section
56 UV-curable cement
58 Scored site
59 Withdrawal process
60 Annular groove with open edge
62 Collar
63 Bending process
64 Discharge nozzle

1. Device for injection of an injectable product, with a storage receptacle that holds the product, a withdrawal arrangement for transferring the product out of the storage receptacle, and an actuation device for applying pressure to the product in the storage receptacle, characterized in that the device (10) can be used selectively as a disposable syringe or as an ampoule for a needle-free injector.

2. Device according to claim 1, wherein the actuating device (16) comprises a plunger rod (26) that has a proximal area (30) that can be separated from a distal area (28).

3. Device according to claim 2, wherein the proximal area (30) is detachably connected to the distal area (28).

4. Device according to claim 2, wherein the proximal area (30) is connected to the distal area (28) via a scored site (32).

5. Device according to claim 1, wherein the withdrawal arrangement (14) comprises a needle (44) whose distal area can be removed in a defined manner from a proximal area of the needle (44) by way of a scored site (58).

6. Device according to claim 5, wherein the proximal area of the needle (44) in an application as an ampoule for a needle-free injector forms a discharge nozzle (64) for the product.

7. Device according to claim 1, wherein the proximal area of the needle (44) has a larger outside diameter and inside diameter than the distal area of the needle.

8. Device according to claim 7, wherein the proximal area has a cylindrical section (50) and a conical section (52) that passes into the discharge nozzle (64).

9. Device according to claim 1, wherein a protective cap (18) is assigned to the withdrawal arrangement (14).

10. Device according to claim 9, wherein the protective cap (18) has a scored site (42) along which a distal end (40) of the protective cap (18) can be removed.

11. Device according to claim 9, wherein a proximal end (38) of the protective cap (18) forms a ring anchor for the storage receptacle (12).

12. Device according to claim 9, wherein the proximal end (38) forms a self-adjusting guide for the device (10) into a needle-free injector.

13. Device according to claim 1, wherein the device (10) consists of materials that are suited for long-term storage of products, especially glass, borosilicate glass, butyl rubber, and high-grade surgical steel.
